SEQUENCE LISTING (14 sequences)

SEQ ID NO: 1 - DNA, 20 bp, Mus musculus
caagggaccctgaagaaggc

SEQ ID NO: 2 - DNA, 24 bp, Mus musculus
ctttggtctttgacttgctcttgg

SEQ ID NO: 3 - DNA, 22 bp, Mus musculus
ctcgaactcaaagcaggagagg

SEQ ID NO: 4 - DNA, 21 bp, Mus musculus
cgtagatggcgaacaggaagg

SEQ ID NO: 5 - DNA, 21 bp, Mus musculus
ttgttgctctggagtccttgc

SEQ ID NO: 6 - DNA, 21 bp, Mus musculus
caggatttcccggacagaagg

SEQ ID NO: 7 - DNA, 20 bp, Mus musculus
acccaacctcgatgtcacca

SEQ ID NO: 8 - DNA, 22 bp, Mus musculus
gcgaggtgaagaattcctctga

SEQ ID NO: 9 - DNA, 21 bp, Mus musculus
ttggcacagtcagtgaggatg

SEQ ID NO: 10 - DNA, 24 bp, Mus musculus
agatttcaagatggaagcaaaggg

SEQ ID NO: 11 - DNA, 24 bp, Mus musculus
actttccataagatggtagcttgg

SEQ ID NO: 12 - DNA, 21 bp, Mus musculus
gcattcacacaccactcaacg

SEQ ID NO: 13 - DNA, 23 bp, Mus musculus
accagaatgaggatcccagaaag

SEQ ID NO: 14 - DNA, 24 bp, Mus musculus
gtagaagctgtaaccatgaggaac

(19) United States
(12) Patent Application Publication
(10) Pub. No.: US 2011/0059102 A1
(43) Pub. Date: Mar. 10, 2011

(54) Therapeutic Method Targeting Midkine

(75) Inventors: Kenji KADOMATSU, Nagoya-shi (JP); Seiichi MATSUO, Nagoya-shi (JP); Yukio YUZAWA, Nagoya-shi (JP)

(73) Assignee: NATIONAL UNIVERSITY CORPORATION NAGOYA UNIVERSITY, Aichi (JP)

(21) Appl. No.: 12/554,560

(22) Filed: Sep. 4, 2009

(51) Int. Cl. A61K 39/395; A61K 31/7088; A61P 9/12; A61P 13/12

(52) U.S. Cl. 424/158.1; 514/44 R

(57) ABSTRACT

Provided is a therapeutic method targeting midkine for treating mammalian kidney disorders, primary kidney disorders, hypertension that follows secondary kidney disorders such as diabetic nephropathy, and hypertension secondary to chronic kidney disease.

### FIELD OF THE INVENTION

This invention relates to a therapeutic method targeting midkine (MK) for a variety of disorders, more specifically to a therapeutic method targeting midkine for kidney disorders, primary kidney disorders, hypertension that follows secondary kidney disorders such as diabetic nephropathy, and hypertension secondary to chronic kidney disease.

### BACKGROUND OF THE INVENTION

Not only does the renin angiotensin system (RAS) contribute to the pathogenesis of hypertension, but also it is an important factor in many cases of internal organ damage, particularly progression of renal damage. This has attracted attention to RAS inhibitors for their role, in addition to treating hypertension, beyond regulation of blood pressure, in protecting internal organs, mainly cardiovascular and renal, ranking them as one of the most frequently used drugs clinically.

Such RAS inhibitors may currently be broadly classified into angiotensin converting enzyme (ACE) inhibitors and AT1 receptor blockers (ARB), which are both RAS inhibitors but differ in efficacy from a standpoint of organ protection; the use of both has been noted to exhibit a synergy effect. Accordingly, there are high expectations for novel drugs with RAS inhibitory function other than the conventional RAS inhibitors, but so far, they have been met only with the prorenin inhibitors currently being studied.

Furthermore, no mechanisms have yet been worked out on chronic kidney disease (CKD) and hypertension secondary to CKD, although they are major disorders, greatly affecting the patient's life prognosis.

Activation of the PAS has conventionally been understood in terms of the systemic RAS activation theory. Namely, it theorizes that the angiotensinogen produced mainly in the liver is converted into angiotensin I by the renin in the kidney, finally into angiotensin II by ACE in the lung, and is coupled with its systemically distributed receptor (AT1R), thereby elevating blood pressure (Systemic RAS activation theory).

However, it has recently been found that all the molecules involved in the above

RAS activation are expressed in the kidney, thereby mainstreaming the local RAS activation theory that RAS is activated locally in the kidney, independently of systemic internal organs, contributing to the onset of hypertension and progression in renal disorder.

This has resulted in less attention being focused on studying systemic RAS activation in a cascade linking many internal organs therein, despite the fact the lung is also an indispensable organ in the RAS activation. Accordingly the current status is one where there is no elucidation of a new RAS activation mechanism via a kidney-lung cross-talk, in particular, with neither a new mechanism for hypertension accompanying CKD nor a new development regarding novel RAS activators.

### SUMMARY OF THE INVENTION

It is an object of the present invention, under such circumstances, to provide a therapeutic method targeting midkine, for various disorders, that is involved in RAS activation, with attention focused on a new RAS activation mechanism.

First, the present invention provides a therapeutic method for treating mammalian kidney disorders, primary kidney disorders, hypertension that follows secondary kidney disorders such as diabetic nephropathy, and hypertension secondary to chronic kidney disease, comprising the step of suppressing midkine expression or activity.

This is based on the present inventors' discovery that midkine is a novel RAS activator that is involved in the activation of ACE, an enzyme important for RAS activation and that suppressing the activity of such midkine can effectively suppress RAS activity. Herein, the kidney disorders include acute kidney failure, chronic kidney failure, end-stage kidney failure, or nephrosclerosis, and the like.

In addition, the present invention includes a method, wherein said step of suppressing midkine expression or activity comprises: the step of preparing a midkine-based nucleic acid drug or a midkine neutralizing antibody; and the step of administering said nucleic acid drug or neutralizing antibody. Use of said nucleic acid drug or neutralizing antibody can effectively suppress midkine activity. Herein, midkine-based nucleic acid drugs or midkine neutralizing antibodies may be locally administered to the lung or kidney.

The present invention may further comprise, in addition to said step of inhibiting the midkine expression or activity, the steps of preparing an angiotensin converting enzyme (ACE) inhibitor or an AT1 receptor blocker (ARB) and administering it. A combined use of a midkine-based nucleic acid drug or a midkine neutralizing antibody with an ACE inhibitor or ARB can more effectively suppress RAS activity.

Further, the therapeutic method of the present invention is applicable to humans.

In addition, the present invention provides a pharmaceutical composition comprising as active ingredients a therapeutically effective amount of a midkine-based nucleic acid drug or a midkine neutralizing antibody, with one or more pharmacologically acceptable diluents, excipients, and/or inert carriers; and such pharmaceutical composition can also be used in combination with an angiotensin converting enzyme inhibitor or an AT1 receptor blocker.

### BRIEF DESCRIPTION OF THE DRAWINGS

FIG. 1: 5/6 nephrectomy induces hypertension and renal damage via the RAS. (A and B) Blood pressure was measured at 0, 2, 4, 6, and 8 weeks after 5/6 nephrectomy. Systolic blood pressure (SBP) (A) and mean blood pressure (MBP) (B) were measured by the tail-cuff method. The mean and SD are represented by squares and bars, respectively, at each time point (Mdk^{+/+}: 0 weeks, n=40; 2 weeks, n=34; 4 weeks, n=19; 8 weeks, n=8; Mdk^{-/-}: 0 weeks, n=26; 2 weeks, n=23; 4 weeks, n=13; 8 weeks, n=4). ***P<0.001 versus Mdk^{-/-} mice. (C) Representative glomerular histology shown by PAS staining. Scale bar: 50 p.m. (D) Semiquantitative analysis of the glomerular sclerosis score. Data are shown as mean and SD (Mdk^{+/+}: 2 weeks, n=5; 4 weeks, n=4; 8 weeks, n=4; Mdk^{-/-}: 2 weeks, n=4; 4 weeks, n=3; 8 weeks, n=3). (E) Plasma Ang II concentration after 5/6 nephrectomy (Mdk^{+/+}: sham, n=6; 2 weeks, n=6; 4 weeks, n=8; Mdk^{-/-}: sham, n=6; 2 weeks, n=7; 4 weeks, n=5). *P<0.05, **P<0.01; †P<0.01 versus sham Mdk^{+/+}. Nx, nephrectomy. (F and G) Effects of hydralazine, temocapril, and olmesartan on blood pressure. SBP (F) and MBP (G) were measured by the tail-cuff method (n=3). ‡P<0.01 versus Mdk^{+/+} mice. (H) Representative histology after treatment with hydralazine, temocapril, and olmesartan. The kidney specimens were stained with PAS. Tubular dilatation (black arrows), tubular cast formation (arrowheads), and tubular degeneration (white arrows) are indicated. Scale bar: 50 μm.

FIG. 2: ACE expression in the lung after 5/6 nephrectomy. (A) ACE protein was determined by Western blotting, and a representative result is shown. The lung tissues were obtained at the indicated time points. (B) Quantitative analysis of ACE protein expression using densitometry. Data are presented as mean and SD (n=3). †P<0.01; ‡P<0.05; *P<0.05 and **P<0.01 versus sham Mdk^{+/+}. (C) ACE mRNA was determined by real-time PCR and normalized to 18S mRNA. Data are presented as mean and SD (Mdk^{+/+}: sham, n=5; 2 weeks, n=5; 4 weeks, n=3; Mdk^{-/-}: sham, n=3; 2 weeks, n=3; 4 weeks, n=3). §P<0.0001; ***P<0.001 and #P<0.0001 versus sham Mdk^{+/+}. (D)

Immunohistochemical staining of lungs with mouse anti-ACE monoclonal antibody at 2 weeks. Upper panel: The first antibody used was anti-mouse ACE monoclonal antibody. Lower panel: Isotype-matched IgG was used as the first antibody. Scale bar: 50 μm. (E) ACE activity was determined by the ACE activity assay, as described in Methods. Data are presented as mean and SD (Mdk^{+/+}: sham, n=5; 2 weeks, n=5; 4 weeks, n=5; Mdk^{-/-}: sham, n=5; 2 weeks, n=5; 4 weeks, n=4). †P<0.01; ‡P<0.05; **P<0.01 versus sham Mdk^{+/+.}

FIG. 3: MK expression in the lung and plasma after 5/6 nephrectomy. (A) Representative data from Western blotting for MK expression in the lung. (B) The intensity of MK bands on Western blotting was normalized to that of β-actin. Data are presented as mean and SD (n=4). *P<0.05 and **P<0.01 versus sham. (C) MK mRNA expression in the lung was determined by real-time PCR and normalized to 18S mRNA. Data are presented as mean and SD (sham, n=5; 2 weeks, n=5; 4 weeks, n=3). ***P<0.001 versus sham. (D) Immunofluorescence staining of MK and thrombomodulin expression in the lung 2 weeks after 5/6 nephrectomy. Lower panels show negative controls using isotype-matched IgG as the first antibody. Arrowhead, bronchial epithelial cells; white arrows, alveolar-capillary endothelial cells. Scale bar: 50 μm. (E) Representative data from Western blotting for MK expression in plasma are shown. Lines under the blot indicate individual samples. (F) Western blot data for plasma MK were quantified using densitometry and are presented as mean and SD (sham, n=4; 2 weeks, n=5). **P<0.01.

FIG. 4: Effects of exogenous MK and PTN in Mdk^{-/-} mice on blood pressure and ACE expression in the lung. (A) SBP was measured by the tail-cuff method at 0 and 2 weeks after 5/6 nephrectomy (n=5). **P<0.01 versus untreated Mdk^{-/-} mice (0 weeks); #P<0.01, rh-MK versus saline. rh-PTN, recombinant human PTN. (B) ACE expression in the lung of Mdk^{-/-} mice treated with MK and saline. Western blot data are shown. (C) The data in B were quantified using densitometry and are presented as mean and SD. †P<0.01. (D) ACE expression in the lung of Mdk^{-/-} mice treated with rh-PTN and saline. Western blot data are shown. (E) The data in D were quantified using densitometry and are presented as mean and SD.

FIG. 5: Mechanism of ACE induction by MK in HMVEC-L. (A) ACE expression in HMVEC-L treated with exogenous MK protein (100 ng/ml) and heparin (20 μg/ml). Lower panels: For PKC inhibition, BIS (5 μM) was added 1 hour before a 36-hour treatment with rh-MK plus heparin. Western blot data are shown, (B) The data in A were quantified using densitometry and are presented as mean and SD (n=3). *P<0.05 and **P<0.01 versus 0 hours. (C) Conversion from Ang Ito Ang II by the cells treated with MK. HMVEC-L cells were treated with exogenous MK protein (100 ng/ml) and heparin (20 μg/ml) or heparin (20 μg/ml) for 36 hours. Ang I (500 μM) was then added to the medium and incubated for the indicated times (n=5). (D) Phospho-PKC expression in HMVEC-L treated with exogenous MK protein (100 ng/ml) and heparin (20 μg/ml). Western blot data are shown, (E) The data in D (left panel) were quantified using densitometry and are presented as mean and SD (n=3). *P<0.05 and **P<0.01 versus 0 hours. (F) Representative data from Western blotting for phospho-PKC expression in the lung. The intensity of phospho-PKC bands on Western blotting was normalized to that of β-actin (n=5). §P<0.05 versus sham Mdk^{+/+}. Western blotting results are not shown. †P<0.01; ‡P<0.05.

FIG. 6: The mechanism of MK induction in the lung. (A) Time course of Nox1, -2, and -4 expression in the lung after 5/6 nephrectomy. Expression of Nox1, -2, and -4 mRNA was determined by real-time PCR. Data are presented as mean and SD (Mdk^{+/+}: sham, n=5; 2 weeks, n=5; 4 weeks, n=3; Mdk^{-/-}: sham, n=3; 2 weeks, n=3; 4 weeks, n=3). *P<0.05, **P<0.01, and ***P<0.001 versus sham Mdk^{+/+}. (B) Effect of tempol on MK expression in the lung. Tempol mixed in drinking water was administered to Mdk^{+/+} mice after 5/6 nephrectomy. MK protein expression was determined by Western blotting (upper panel). The intensity of MK bands on Western blotting was normalized to that of β-actin (lower panel). Data are presented as mean and SD (n=5). (C) Effect of tempol on blood pressure after 5/6 nephrectomy. SBP was measured by the tail-cuff method. Data are presented as mean and SD (n=6). (D) Effect of tempol on renal damage. PAS staining of a kidney specimen 2 weeks after 5/6 nephrectomy in Mdk^{+/+} mice that received tempol treatment. The tubulointerstitial damage was slight compared with that in the mice without tempol treatment (FIG. 1C). Scale bar: 20 μm. †P<0.05; ‡P<0.01.

FIG. 7: Schematic diagram showing the possible relationship between MK and the RAS cascade that promotes hypertension and renal damage.

### DETAILED DESCRIPTION OF THE INVENTION

Midkine (MK), a target in the therapeutic method related to the present invention, is a 13 kDa molecular weight, low molecular retinoic acid-derived heparin bound type protein.

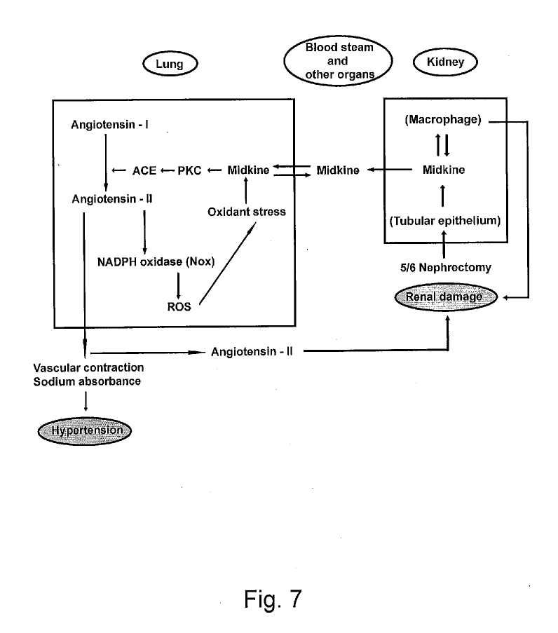

The present inventors studied such midkine as an important factor linking the kidney to the lung, and elucidated that midkine is a novel RAS activator involved in ACE activation. The present inventors further established a new concept designated as a “midkine-mediated kidney-lung crosstalk” with respect to inter-organ interactions in CKD. As exhibited in FIG. 7, it was confirmed that blood pressure elevation due to CKD is completely midkine dependent and that the expression of midkine in the lung rises from oxidative stress induced by renal damage, thereby evoking enhanced ATII production and systemic hypertension through the ACE activation in the lung. Moreover, administration of midkine to midkine-deficient mice restores blood pressure elevation, revealing that midkine is a causal factor for the onset of hypertension in CKD disease conditions. Moreover elevated oxidative stress (ROS) resulting from the RAS activation in the lung promotes MK expression in the lung, thereby creating a vicious cycle involving the RAS activation in the lung. It is conceptualized that as a consequence, the elevated production of angiotensin II and hypertension further accelerate progression of renal damage.

Among the methods for targeting such midkine to suppress the activity thereof are, for example, one comprising preparing a midkine-based nucleic acid drug or a midkine neutralizing antibody; and administering said nucleic acid drug or neutralizing antibody. Nucleic acid drugs include a short interfering RNA (siRNA) corresponding to a specific base sequence of a midkine messenger RNA, and the like. Such siRNA may be designed and synthesized, for example, based on the sequence selection rule, from within the translated region of human MK mRNA (GenBank Accession No. M34327: Tomomura M. et al.: J. Biol. Chem., 265:10765-10770, 1990). Such midkine-based nucleic acid drug or neutralizing antibody may be administered locally to a site afflicted with a disorder, or administered systemically. The method of administration may be oral administration, transdermal administration, or any other method of administration.

Note that the above midkine-inhibiting nucleic acid drug or neutralizing antibody may also be used in combination with a conventional known RAS inhibitor. Such RAS inhibitors include an ACE converting enzyme inhibitor, an AT1 receptor blocker (ARB), and the like.

When a therapy targeting midkine is contemplated, depending on the differences in disease conditions and the organs, cases exist where midkine is involved in organ damage and also where it is involved in organ protection. The former cases include cancer, inflammation (angiostenosis lesions after endothelial cell damage, acute kidney disorder, diabetic nephropathy, rheumatic disease, post surgery adhesion lesions, and the like), kidney disorder, primary kidney disorder, hypertension that follows secondary kidney disorders such as diabetic nephropathy, and hypertension secondary to chronic kidney disease. The latter cases include neuronal protection and myocardial preservation effects in cerebral infarction and myocardial infarction, which are considered to be due to the anti-apoptotic action of midkine. Thus, there arise two clinical applications; an anti-midkine therapy which suppresses the midkine's function and a midkine replacement therapy anticipating midkine's organ protective action.

Anti-Midkine Therapy

Midkine that is newly induced to be expressed is highly involved in the onset of disorders such as cancer, inflammation (angiostenosis lesions after endothelial cell damage, acute kidney disorder, diabetic nephropathy, rheumatic disease, post surgery adhesion lesions, and the like), kidney disorder, primary kidney disorder, hypertension that follows secondary kidney disorders such as diabetic nephropathy, and hypertension secondary to chronic kidney disease; and the efficacy of an anti-midkine therapy has been demonstrated at respective animal experiment levels and its clinical application is anticipated.

The enhanced expression of midkine has been confirmed in nearly all cases of cancer and it has been reported that the midkine levels in the blood in several cancers such as neuroblastoma and bladder cancer are useful tumor markers, as they correlate to the patient's life prognosis. Nude mice inoculated with human colorectal carcinoma [cells], upon administration of midkine antisense ODN (oligodeoxynucleotide), were confirmed to have shrinkage of their tumors (Cancer Res. 61: 8486-8491 2001). Additionally, the efficacy of an anti-tumor therapy using an adenovirus (Ad5MK) containing a midkine promoter was demonstrated in vitro (J Exp Clin Cancer Res 27:1-13, 2008).

Further, it has been reported that midkine induces EMT (Epithelial-mesenchymal transition) through Notch2/Jak2-Stat3 signaling in cancer cells (Cell Cycle 7:1613-1622, 2008). Since EMT□ is also important in fibrosis, it is conceivable to apply an anti-midkine therapy not only to blocking carcinogenesis and metathesis, but also to treating fibrosis involved in renal failure, hepatocirrhosis, interstitial pneumonia, and the like.

On the other hand, in the case of inflammation, midkine has migratory activity for inflammatory cells such as neutrophils, macrophages, and the like, and smooth muscle cells; knocking down midkine in animal models for vasculare restenosis, nephritis (ischemic reperfusion model and cisplatin renal disorder), and rheumatoid arthritis was noted to be effective for improving the disease conditions. Further, in addition to its direct migratory effect on inflammatory cells, midkine also induces chemokine production such as MCP-1 from the tubular epithelial cells, such that the efficacy of an anti-midkine therapy not only provides a direct effect, but it also suppresses an indirectly induced chemokine production (J Immunol 167:3463-3469, 2001).

Another interesting disease is diabetes mellitus; in particular, microinflammation is important for the pathogenesis and progression of diabetic nephropathy. Hyperglycemia or oxidative stress induces midkine to be expressed from mesangial cells or tubular epithelial cells and to be deeply involved in a local macrophage infiltration. Midkine was therein found to be involved not only in the activation of ERK (extracellular signal-regulated kinase) but also in the activation of PEK (Protein kinase C) into glomerular sclerosis (Am J Pathol. 168: 9-19 and 2006). It was recently confirmed that hypertension accompanying CKD is midkine dependent and that midkine evokes increased production of ATII and systemic hypertension through ACE activation in the lung; it is conjectured that the ACE activation by midkine also occurs through PKC Clin Invest. 119(6): 1616-1625 and 2009). Suppression of PKC activation by an ant-midkine therapy offers a possible application thereof for treating diabetic complication and hypertension among others.

Midkine Replacement Therapy

Unlike the preceding disorders, midkine provides cytoprotective effects, particularly in the neural region. In a constant light-induced retinal nerve degeneration model, a prior injection of midkine reduced a neuronal cell loss. Further, in a cerebral ischemia reperfusion insult model, unlike renal ischemia reperfusion, prior ventricular administration of midkine was able to remedy the loss of hippocampal neuronal cells (Brain Res. 9:894:46-55, 2001). In this case, midkine administration immediacy before an ischemia reperfusion insult is critical, but post-insult therapeutic administration is ineffective. However, a reduction in cerebral infarction was noted as a result of postischemic gene transfer of midkine into the brain (Gene Therapy 12:487-493, 2005.), where the timing of administration, administration procedure, expression duration, and the like are important.

Similarly, a reduction in the apoptotic loss of myocardial cells has also been noted with administration of midkine (Am J Physiol Heart Circ Physiol 296:H462-H469) in a model of acute myocardial infarction caused by coronary ischemic reperfusion injury (Circulation 114:1713-1720, 2006).

### EXAMPLES

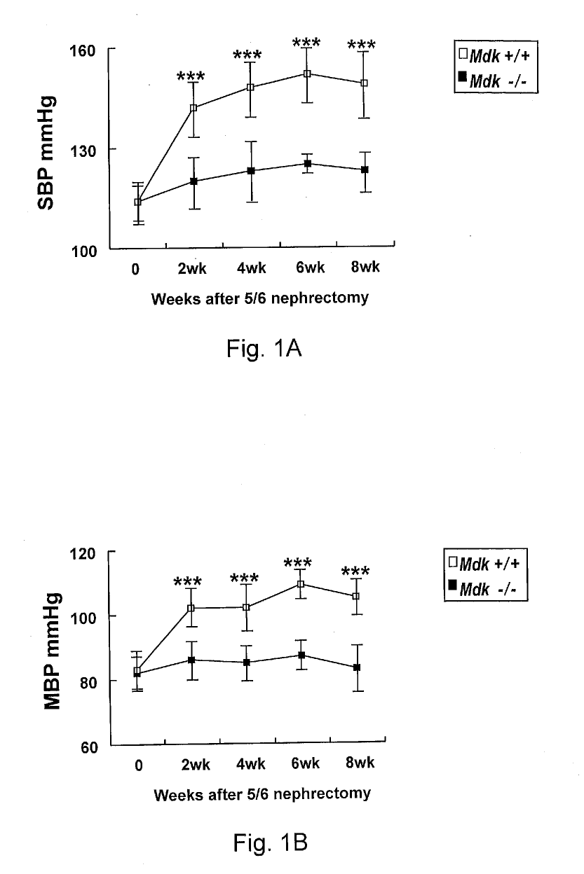

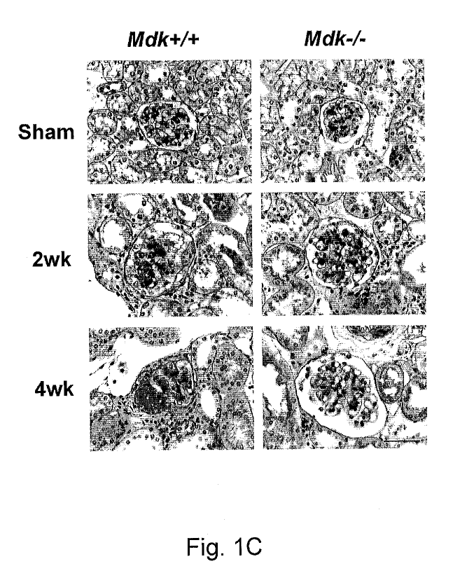

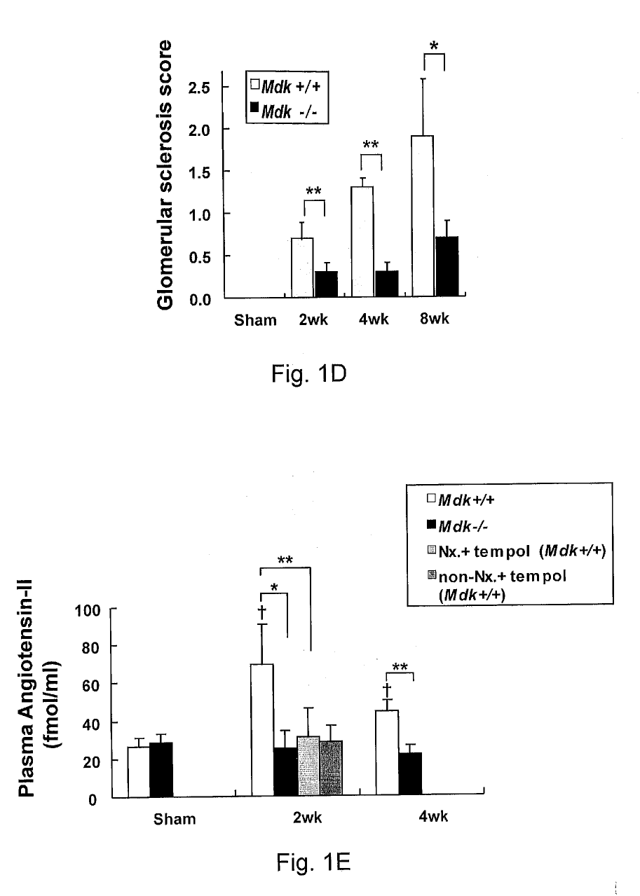

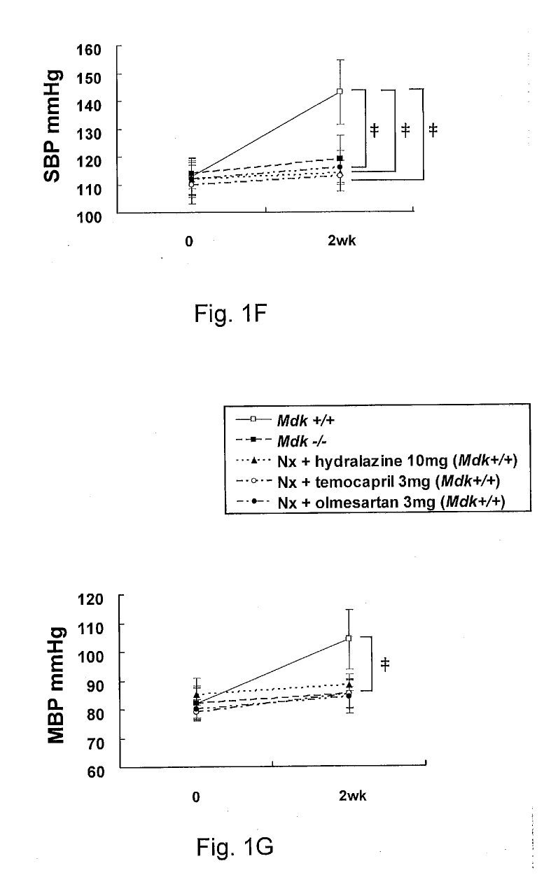

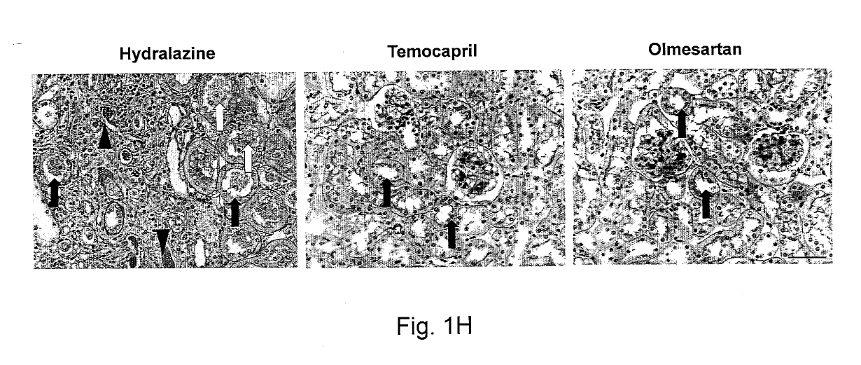

MK is involved in RAS activation induced by 5/6 nephrectomy. Systolic and mean blood pressure were comparable in untreated Mdk^{+/+} and Mdk^{-/-} mice (FIGS. 1, A and B). However, we found that 5/6 nephrectomy strikingly increased blood pressure in Mdk^{+/+} mice but not in Mdk^{-/-} mice (FIGS. 1, A and B). The systolic and mean blood pressure of Mdk^{+/+} mice strikingly increased after 2 weeks, but Mdk^{-/-} mice showed almost normal blood pressure, i.e., no significant increase (systolic blood pressure, 143±11.6 mmHg in Mdk^{+/+} mice vs. 119±8.6 mmHg in Mdk^{-/-} mice; mean blood pressure, 104±10.3 mmHg vs. 85±6.7 mmHg). Consequently, systolic and mean blood pressures were significantly higher in Mdk^{+/+} than in Mdk^{-/-} mice from 2 to 8 weeks (FIGS. 1, A and B).

5/6 nephrectomy caused not only hypertension but also progressive renal failure. Blood urea nitrogen and serum creatinine levels gradually increased, and both parameters were significantly higher in Mdk^{+/+} mice at 2 and 4 weeks after renal ablation (Table 1).

TABLE 1
Body weight, blood urea nitrogen, serum creatinine,
and left kidney weight after 5/6 nephrectomy

| | BW (g) | BUN (mg/dl) | Cre (mg/dl) | Left kidney wt (mg) |
|---|---|---|---|---|
| Before nephrectomy | | | | |
| Mdk^{+/+} | 22.2 ± 2.1 | 21.2 ± 3.7 | 0.06 ± 0.02 | — |
| Mdk^{-/-} | 21.3 ± 1.3 | 19.7 ± 1.4 | 0.04 ± 0.02 | — |
| Hydralazine, 10 mg/kg/d | 23.1 ± 0.6 | ND | ND | — |
| Temocapril, 3 mg/kg/d | 22.0 ± 2.0 | ND | ND | — |
| Olmesartan, 3 mg/kg/d | 24.5 ± 0.5 | ND | ND | — |
| Tempol, 3 mmol/l | 22.7 ± 1.7 | ND | ND | — |
| 2 weeks after nephrectomy | | | | |
| Mdk^{+/+} | 19.7 ± 2.2 | 57.1 ± 9.7 | 0.65 ± 011 | 104.8 ± 9.6 |
| Mdk^{-/-} | 18.8 ± 1.5 | 39.5 ± 7.2^{A} | 0.39 ± 0.08^{A} | 104.3 ± 15.1 |
| Hydralazine, 10 mg/kg/d | 18.7 ± 0.7 | 63.3 ± 7.6 | 0.53 ± 0.12 | ND |
| Temocapril, 3 mg/kg/d | 19.7 ± 2.7 | 30.3 ± 2.9^{A} | 0.21 ± 0.04^{A} | ND |
| Olmesartan, 3 mg/kg/d | 21.3 ± 0.7 | 36.0 ± 1.0^{B} | 0.21 ± 0.01^{A} | ND |
| Tempol, 3 mmol/l | 21.4 ± 1.6 | 41.2 ± 3.9^{B} | 0.25 ± 0.04^{A} | ND |
| 4 weeks after nephrectomy | | | | |
| Mdk^{+/+} | 20.9 ± 1.8 | 66.1 ± 8.7 | 0.77 ± 0.13 | 130.0 ± 21.4 |
| Mdk^{-/-} | 20.6 ± 1.7 | 46.4 ± 4.8^{A} | 0.44 ± 0.15^{B} | 130.3 ± 10.7 |
| 8 weeks after nephrectomy | | | | |
| Mdk^{+/+} | 22.5 ± 1.9 | 71.9 ± 14.6 | 1.56 ± 0.34 | 118.5 ± 14.9 |
| Mdk^{-/-} | 21.2 ± 2.3 | 62.9 ± 12.9 | 1.18 ± 0.29 | 128.5 ± 6.0 |

Values are mean = SD.
BUN, blood urea nitrogen; Cre, serum creatinine. ND, no data.
Hydralazine, temocapril, olmesartan, and tempol were administered to Mdk^{+/+} mice at the indicated doses.
^{A}P < 0.001 verses Mdk^{+/+}.
^{B}P < 0.01 versus Mdk^{+/+}.

Mdk^{+/+} mice also exhibited more severe glomerular sclerosis, which is characterized by a marked deposition of extracellular matrix in the glomeruli and which occurred as early as 2 weeks after renal ablation (FIG. 1C). Semiquantitative analysis of the glomerular sclerosis scores revealed significant differences between Mdk^{+/+} and Mdk^{-/-} mice (FIG. 1D). In addition, the tubulointerstitial damage was worse in Mdk^{+/+} mice (Supplemental FIG. 1; supplemental material available online with this article; doi:10.1172/JCI37249DS1). Thus, tubular dilatation, cast formation in the tubular lumen, and tubular epithelial degeneration became apparent at 2 weeks after renal ablation and were more severe in Mdk^{+/+} mice (Supplemental FIG. 1, A-H). Interstitial fibrosis, as evidenced by collagen deposition revealed by Masson's trichrome staining, was exhibited at 4 weeks and then more diffusely at 8 weeks, and the stained area became expanded in Mdk^{+/+} mice (Supplemental FIG. 1, I-P). These data collectively indicate that renal damage was more severe in Mdk^{+/+} mice than in Mdk^{-/-} mice.

These symptoms of hypertension and renal damage were attributable to the RAS, as (a) the hypertension was accompanied by elevated plasma Ang II concentration (FIG. 1E); and (b) the ACE inhibitor temocapril and the angiotensin receptor blocker olmesartan reduced both blood pressure and renal tubulointerstitial damage, but the vasodilator hydralazine only ameliorated hypertension (FIG. 1, F-H). Abnormal elevation of blood urea nitrogen and serum creatinine was ameliorated by administration of temocapril and olmesartan, but not hydralazine (Table 1). It is therefore conceivable that the RAS contributed to both hypertension and renal damage.

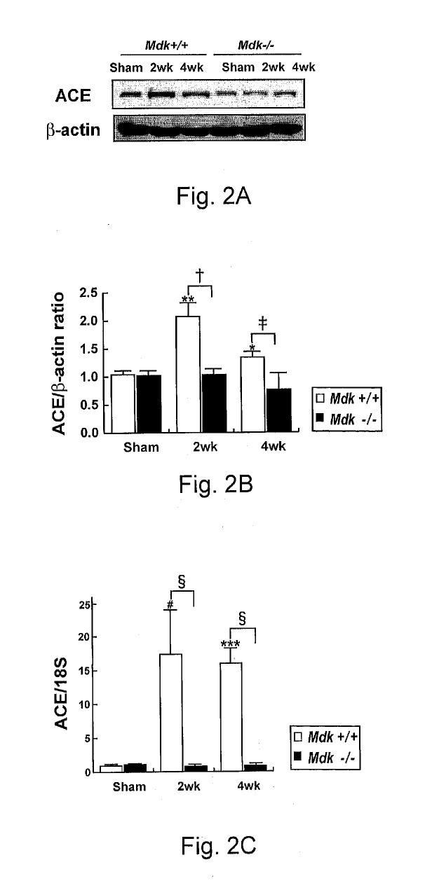

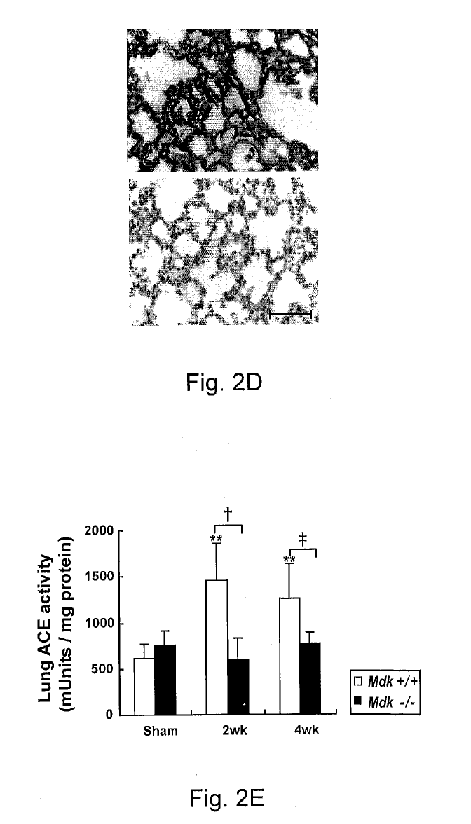

ACE levels are increased in the lung after 5/6 nephrectomy. Expression of the intrarenal angiotensinogen and renin was suppressed and ACE expression was unchanged in Mdk^{+/+} mice, which was consistent with previous findings after 5/6 nephrectomy of rats (Am. J. Physiol. 263:F731-F738, Acta Physiol. (Oxf.). 189:369-377) (Supplemental FIG. 2, A-C). In contrast to the kidney, the lung showed significant increases in ACE expression and its activity 2 and 4 weeks after renal ablation (FIG. 2, A-E). ACE protein expression was localized to the pulmonary vascular endothelial cells and alveolar-capillary endothelial cells, consistent with a previous report (FIG. 2D) (Genome Biol. 4:225).

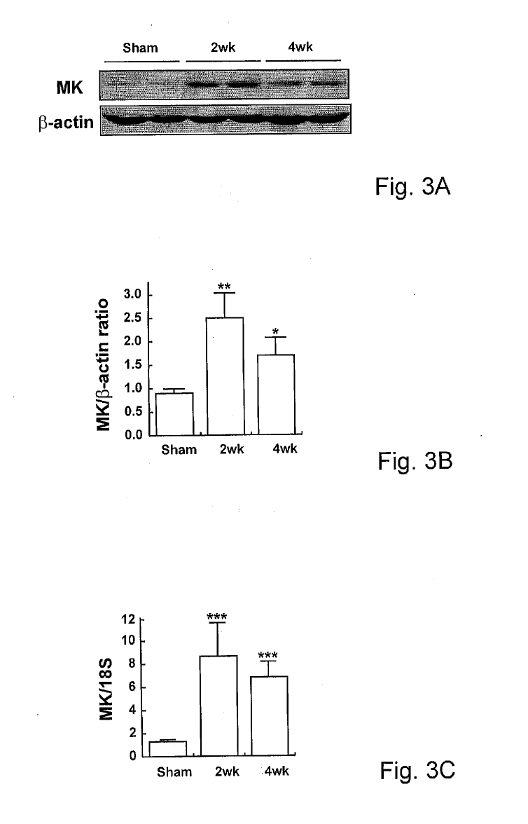

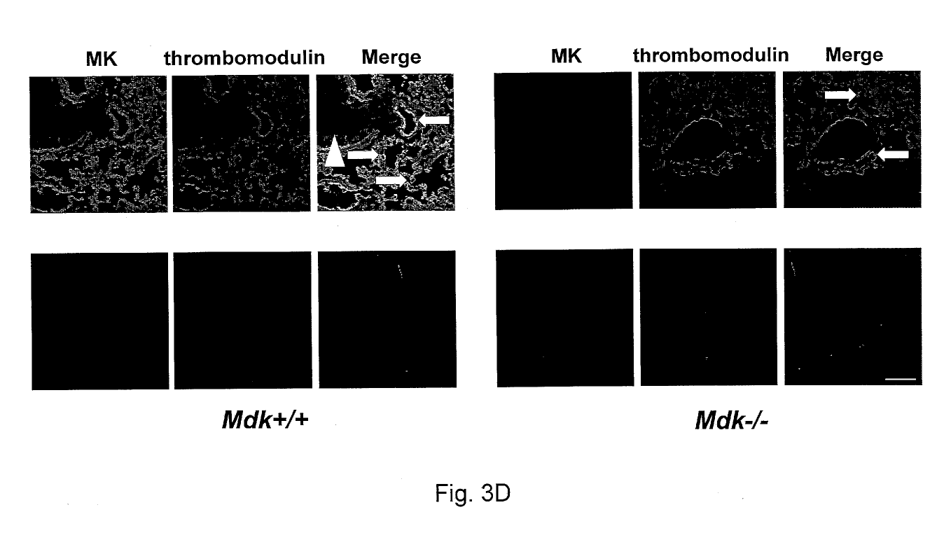

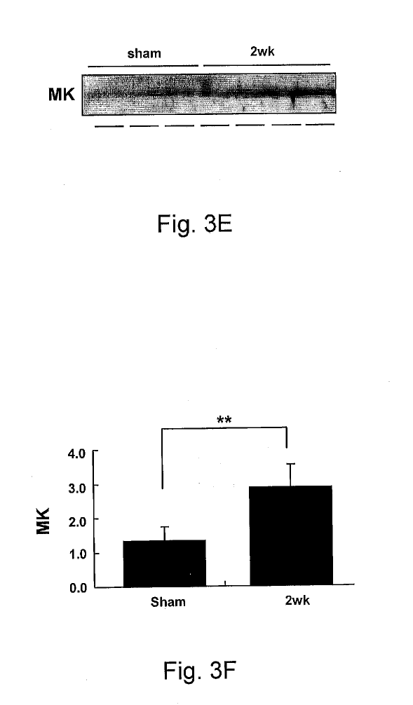

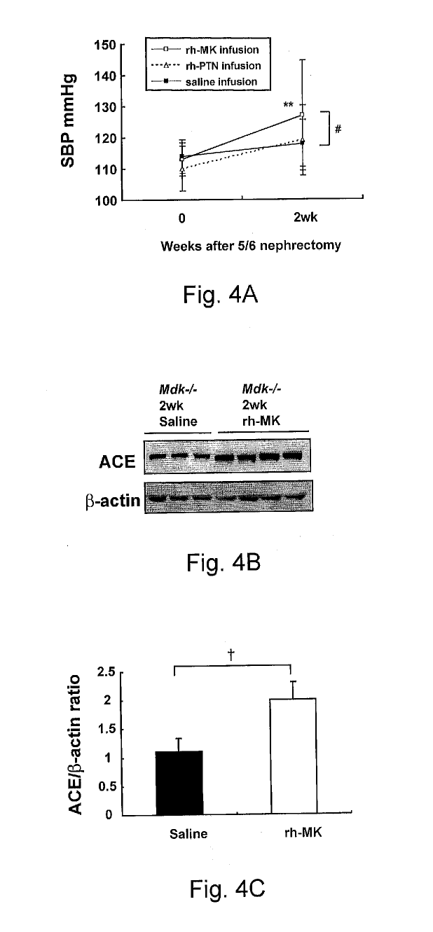

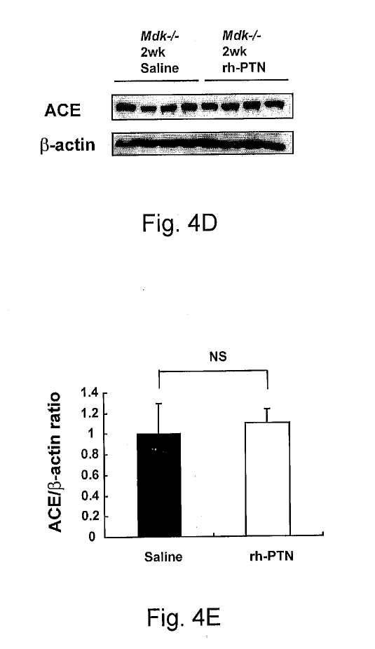

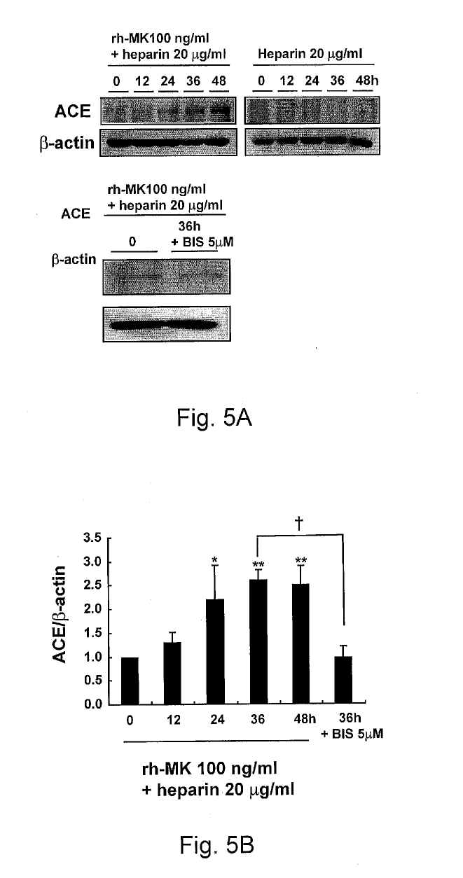

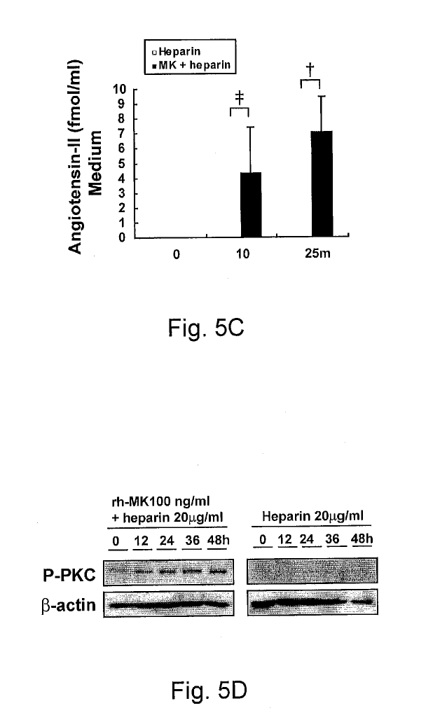

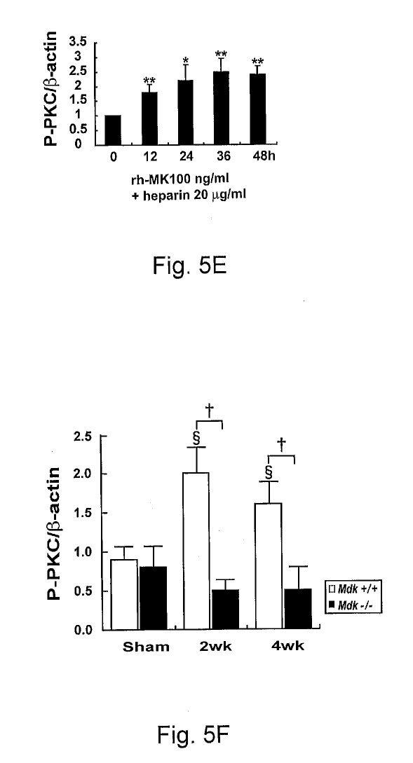

Other organs that play major functions in the RAS were also examined for their expression of RAS components. Angiotensinogen mRNA expression was induced in the liver after 5/6 nephrectomy, but the expression level was not significantly different in Mdk^{+/+} and Mdk^{-/-} mice (Supplemental FIG. 3A). There was no difference in angiotensinogen protein levels between the two genotypes (Supplemental FIGS. 3, B and C), and renin mRNA expression was not detected in the liver (data not shown). Expression of ACE protein and MK protein was also not detected in the liver (Supplemental FIGS. 3, D and E). In the brain, mRNA expression of angiotensinogen and ACE did not change after 5/6 nephrectomy (Supplemental FIGS. 4, A and B), and the renin mRNA expression was undetectable (data not shown). ACE protein was also not detected in the brain (Supplemental FIG. 4C). Furthermore, the mRNA expression of angiotensinogen, renin, and ACE did not change in the heart (Supplemental FIG. 5). Therefore, it is most likely that the hypertension observed after 5/6 nephrectomy was due to activation of the lung ACE.

MK levels are increased in the lung, kidney, and plasma after 5/6 nephrectomy. MK expression was increased in the lung in association with an elevation in ACE expression (FIG. 3, A-C). MK protein was localized to the endothelium of microvessels of the lung, as revealed by the use of thrombomodulin as a marker of the vascular endothelium (FIG. 3D). MK expression was detected in alveolar-capillary endothelial cells but not in bronchial epithelial cells (FIG. 3D).

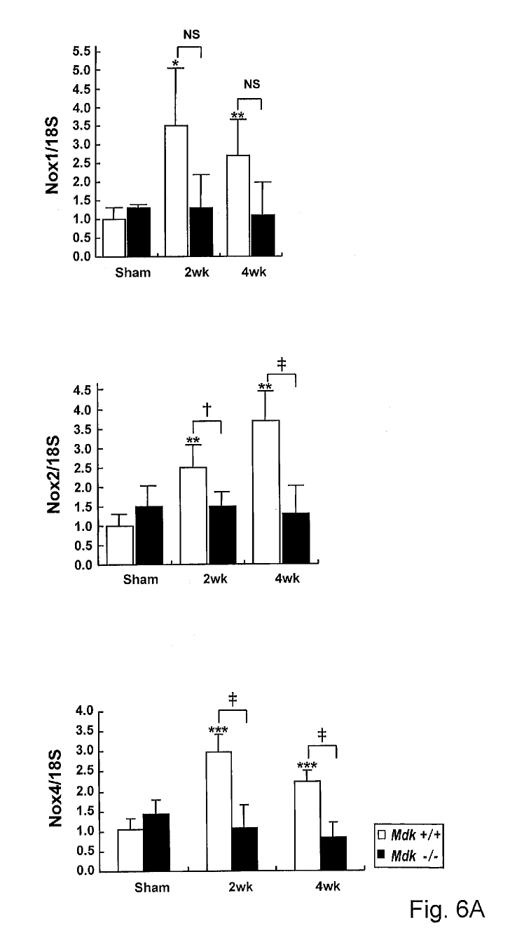

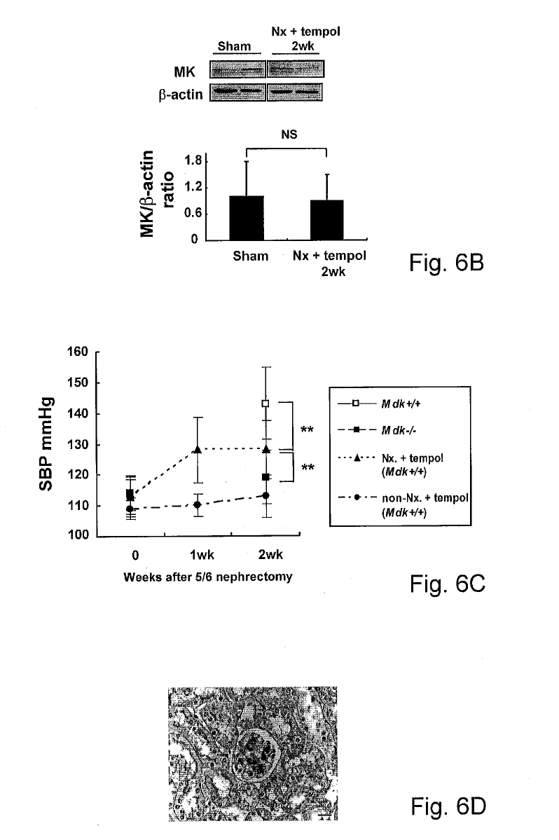

Histological evidence of lung damage, i.e., due to edema and degeneration of alveolar cells, was not observed after 5/6 nephrectomy in the Mdk^{+/+} and Mdk^{-/-} mice (Supplemental FIG. 6A). Increases in macrophage and neutrophil infiltration into the lung were also not observed after 5/6 nephrectomy in the two genotypes (Supplemental FIG. 6, B-E). These results indicated that the increase in MK expression in the lung after 5/6 nephrectomy was not due to leukocytes.

MK expression was also significantly elevated in the kidney at both the protein and mRNA levels 2 and 4 weeks after renal ablation (Supplemental FIG. 7, A-C). Immunohistochemical analysis revealed that MK protein was mainly localized in the tubular epithelium (Supplemental FIG. 8A). This result is consistent with previous reports in which MK was expressed in the kidney after ischemia/reperfusion injury and its associated massive leukocyte infiltration (J. Immunol. 167:3463-3469). We also detected a substantial increase in macrophage infiltration into the kidney after 5/6 nephrectomy, and this increase was significantly higher in Mdk^{+/+} mice than Mdk^{-/-} mice (Supplemental FIGS. 8, B and C). It is known that MK is expressed by activated macrophages (J. Vase. Surg. 47:1322-1329, Biochem. Biophys. Res. Commun. 317:108-113). Thus, it is conceivable that the increase in MK expression in the kidney after 5/6 nephrectomy was due to enhanced expression in both the tubular epithelium and infiltrating macrophages.

Since MK is a secreted protein, and its expression was induced by 5/6 nephrectomy in the kidney and lung (FIGS. 3, A-C, and Supplemental FIGS. 7, A-C), we next examined plasma MK levels. As shown in FIGS. 3, E and F, plasma MK levels were indeed increased 2 weeks after 5/6 nephrectomy.

Exogenous MK induces ACE expression. If MK is required for ACE expression in the lung and hypertension, supplementary administration of MK might also affect these symptoms. To clarify this issue, we administered exogenous MK continuously through an osmotic pump into Mdk^{-/-} mice after 5/6 nephrectomy. This administration was found to restore hypertension and ACE expression in the lung (FIG. 4, A-C). We also administered pleiotrophin (PTN; also called HB-GAM); which shows 50% homology with MK (Cancer Lett. 204:127-143), to Mdk^{-/-} mice after 5/6 nephrectomy. However, exogenous PTN neither induced hypertension nor increased ACE expression in the lung (FIGS. 4, A, D, and E). These data support the specificity of MK with respect to its involvement in ACE expression and blood pressure regulation.

Furthermore, exogenous MK protein on primary cultured human lung microvascular endothelial cells (HMVEC-L) significantly enhanced ACE expression, suggesting that ACE is one of the targets of MK in the lung (FIGS. 5, A and B). When Ang I was added to the culture medium of the lung endothelial cells treated with MK and heparin for 36 hours, Ang I was converted to Ang II in a time-dependent manner, while cells treated with heparin alone did not show such a conversion (FIG. 5C). These results suggest that MK is a potent inducer of Ang II through upregulation of ACE expression in lung endothelial cells.

Along with the increase in ACE expression, phosphorylation levels of PKC were also increased in primary cultured HMVEC-L after exposure to exogenous. MK (FIGS. 5, D and E). This result suggests that MK upregulates ACE expression through activation of PKC. This idea was further supported by three lines of evidence. First, bisindolylmaleimide I (BIS), a PKC-specific inhibitor, blocked the MK-mediated increase in ACE expression (FIGS. 5, A and B). Second, PKC phosphorylation was significantly increased in the lungs of Mdk^{+/+} but not Mdk^{-/-} mice after 5/6 nephrectomy (FIG. 5F). Third, consistent with previous reports (J. Biol. Chem. 273:25191-25197), the increase in ACE expression in primary cultured HMVEC-L was also induced by PMA, a PKC activator, and was blocked by BIS (Supplemental FIG. 9).

Oxidant stress induces MK expression in the lung after 5/6 nephrectomy. Finally, the mechanism of MK induction in the lung by 5/6 nephrectomy was investigated. The NADPH oxidases (Nox's) are superoxide-generating enzymes that release superoxide by electron transfer from NADPH to oxygen. Increased production of ROS has been implicated in various pathologies, including hypertension, atherosclerosis, diabetes, and CKD (Expert Rev. Cardiovasc. Ther. 1:91-106, Nat. Clin. Pract. Nephrol. 2:582-593). In the present study, Nox1, -2, and -4 mRNA expression in the lungs of Mdk^{+/+} mice was found to be significantly increased at 2 and 4 weeks after renal ablation compared with the levels in the sham-operated animals, suggesting that oxidant stress was generated in the lung; in contrast, the expression of Nox1, -2, and -4 mRNA was unchanged in the Mdk^{-/-} mice (FIG. 6A). A cell membrane-permeable radical scavenger, 4-hydroxy-2,2,6,6-tetramethylpiperidine-N-oxyl (tempol), reduced MK expression to normal levels in the lung (FIG. 6B); plasma Ang II levels (FIG. 1E) and blood pressure (FIG. 6C) were also reduced. Tempol also ameliorated glomerular sclerosis and tubulointerstitial damage (FIG. 6D) and improved renal function, i.e., significantly reduced blood urea nitrogen and serum creatinine levels (Table 1). These results suggest that MK expression was induced by oxidative stress in the lung after 5/6 nephrectomy. Tempol also reduced MK expression in the kidney (Supplemental FIGS. 7, D and E).

### <Discussion>

Our study demonstrated that Mdk^{-/-} mice had almost normal blood pressure after 5/6 nephrectomy, while wild-type mice showed marked hypertension. This hypertension was ameliorated by RAS-related inhibitors and indeed was accompanied by elevated plasma Ang II levels. Surprisingly, ACE activity was enhanced in the lung, whereas RAS components were not activated in other organs. Plasma MK levels and MK expression in the lung and kidney were elevated. Supplementary MK administration to Mdk^{-/-} mice restored lung ACE expression and hypertension. MK also induced ACE expression and consequently conversion from Ang Ito Ang II in primary cultured lung microvascular endothelial cells. We therefore concluded that MK-mediated ACE induction in the lung is critical for hypertension induced by 5/6 nephrectomy (FIG. 7).

Inter-organ interactions involving the kidney have recently been highlighted. Regarding factors affecting lung function after acute kidney injury, several cytokines, including IL-6, IL-1β, and TNF-α, have been suggested as candidates (J. Am. Soc. Nephrol. 18:155-164, J. Am. Soc. Nephrol. 14:1549-1558). Such results contribute to our understanding of the high mortality associated with pulmonary complications following acute kidney injury. CKD has also been linked with damage in other organs, especially with cardiovascular damage (so-called cardiorenal syndrome) (N. Engl. J. Med. 351:1285-1295). It is particularly interesting that RAS components are increased in the heart and brain of subtotally nephrectomized rats (Acta Physiol. (Oxf.). 189:369-377, J. Am. Soc. Nephrol. 17:2112-2119) and that Ang II amounts are increased in the isolated perfused hind limbs of uremic rats (J. Hypertens. 9:41-48). Inhibitors of the RAS, e.g., angiotensin receptor blockers and ACE inhibitors, are indeed the first choice of therapy for CKD (Am. J. Kidney Dis. 43 (5 Suppl. 1):S1-S290). Our results clearly show that the lung is a promising target in the cross-talk between the kidney and other organs and suggest that MK is a candidate mediator for pulmonary and other organ complications associated with CKD.

Regarding the cross-talk between the kidney and lung in 5/6 nephrectomy, our study has also provided an insight into the underlying mechanism. We found that Nox1, -2, and -4 were induced in the lung and that tempol reduced MK and plasma Ang II levels. Nox mediates the initial reaction of 3 successive reduction products of molecular oxygen, i.e., superoxide (O2.-), hydrogen peroxide (H2O2), and hydroxyl radical (OH.). Since tempol is a membrane-permeable and metal-independent superoxide dismutase mimetic that is specific for superoxide anion (O2.-) (Biochem. Pharmacol. 38:2581-2586, Hypertension. 33:424-428), tempol may target ROS initiated by Nox in the lung. To the best of our knowledge, this is the first study to show that 5/6 nephrectomy induces oxidative stress in the lung. We have previously reported that oxidative stress induces MK expression (Am. J. Pathol. 168:9-19, J. Immunol. 167:3463-3469). Therefore, it is conceivable that Nox-mediated ROS production leads to an induction of MK expression in the lung (FIG. 7). As ROS have very short half-lives (Mol. Aspects. Med. 26:340-352), it is not likely that the ROS themselves travel between the kidney and lung. Furthermore, it is widely accepted that Ang II induces Nox expression (Circ. Res. 90:E58-E65.). Based on this background data, we speculate that the following molecular circuit is established after circulating MK induces ACE expression in the lung: Ang II induces Nox expression, which in turn initiates ROS production and subsequently MK and ACE expression (FIG. 7).

RAS-related inhibitors, but not hydralazine, ameliorated renal damage in the present study. It has also been reported that systemically administered Ang II worsens renal function (Hypertension. 52:256-263, Hypertension. 43:1126-1132, Am. J. Physiol. Renal Physiol. 294:F161-F169). Therefore, the RAS might play at least a partial role in renal damage. In addition, we found a significant elevation of macrophage infiltration into the kidney in wild-type mice compared with Mdk^{-/-} mice in the present study. This finding is consistent with our previous report showing MK-mediated macrophage recruitment in neointima formation (J. Clin. Invest. 105:489-495). Therefore, MK-mediated inflammation in the kidney may contribute to the renal damage after 5/6 nephrectomy (FIG. 7). On the other hand, in the context of inflammation and hypertension, it is important to note that inflammatory cell infiltration into the kidney does not always induce hypertension (J. Am. Soc. Nephrol. 19:77-83, Diabetes 56:363-372). Furthermore, the local RAS in organs other than the lung was not activated in the present study. Thus, the relationship between intrarenal inflammation and hypertension after 5/6 nephrectomy remains to be verified.

### Methods

Animals and experimental design. Mice deficient in Mdk were generated as described previously (Genes Cells. 3:811-822). After backcrossing of Mdk+/− mice for 14 generations with 129/Sv mice, Mdk+/− mice were mated with each other to generate Mdk^{+/+} and Mdk^{-/-} mice, which were used in this study. Experiments were performed on 8 to 12-week-old female mice weighing 20-25 g that were housed under controlled environmental conditions and maintained with standard food and water. Animal care and experimental procedures were approved by the Animal Experimentation Committee of the Nagoya University Graduate School of Medicine and were conducted according to the Nagoya University Regulations for Experiments. Renal ablation was performed as described previously. Briefly, mice were anesthetized by diethyl ether. The flank region was shaved, and the animals were placed on a heating pad to maintain a constant body temperature (37° C.). Under anesthesia, right flank incisions were made, and the right kidney was removed. Seven days later, mice were anesthetized as above, and two-thirds of the mass of the left kidneys was ablated (Mdk^{+/+}: 2 weeks, n=13; 4 weeks, n=13; 8 weeks, n=8; Mdk^{-/-}: 2 weeks, n=10; 4 weeks, n=10; 8 weeks, n=4). After the renal ablation, the flanks were closed in 2 layers with 5-0 silk sutures. The animals received 100 ml/kg warm saline instilled into the peritoneal cavity during the procedure and were allowed to recover with free access to food and water. In the control mice, a sham operation was performed (Mdk^{+/+}, n=11; Mdk^{-/-}, n=8).

Drug treatment model. In the drug treatment studies, Mdk^{+/+} mice were randomly assigned to receive one of the following 5 treatments after 5/6 nephrectomy and were orally treated daily starting from 24 hours after 5/6 nephrectomy: vasodilator (hydralazine hydrochloride; Novartis) at a dose of 10 mg/kg/d (group 1, n=3); ACE inhibitor (temocapril; Daiichi Sankyo Co.) at a dose of 3 mg/kg/d (group 2, n=3); angiotensin receptor blocker (olmesartan; Daiichi Sankyo Co.) at a dose of 3 mg/kg/d (group 3, n=3); antioxidative reagent (tempol; Alexis Biochemicals) at a dose of 3 mmol/l in drinking solution after 5/6 nephrectomy (group 4, n=6); and sham operation (group 5, n=6). Drug treatment was performed at 2 weeks after 5/6 nephrectomy. In the present study, we determined the dosage according to previous reports (Hypertension. 13:582-588, J. Cardiovasc. Pharmacol. 40:533-542, Hypertension. 43:841-848). Blood pressure was measured by the tail-cuff method at 2 weeks, as described below under Blood pressure monitoring. Mice were sacrificed at 2 weeks after 5/6 nephrectomy, and blood and tissue samples were collected.

MK protein and PTN protein. The recombinant human MK (rh-MK) was produced from yeast as described previously (Br. J. Cancer. 83:701-706). In brief, human MK was produced by Pichia pastoris GS115 transfected with a human MK expression vector, which was constructed into pHIL-D4 (Invitrogen) (Br. J. Cancer. 83:701-706). The MK protein was then purified by anion exchange chromatography and affinity chromatography on a heparin column. The purified human protein exhibited neurotrophic activity comparable to that of mouse MK produced in L cells (Biochem. Biophys. Res. Commun. 177:652-658). Recombinant human PTN was produced as previously described (Biosci. Biotechnol. Biochem. 67:2288-2290).

rh-MK and -PTN treatment model. In the pump study, MK protein in saline (1.6 mg/ml) (n=5), PTN in saline (1.6 mg/ml; n=5), or saline alone (n=5) was infused using an osmotic pump (ALZA Corp.) into Mdk^{-/-} mice after 5/6 nephrectomy. The pumps continuously infused a total of 100 μl over 14 days. The pumps were implanted under the dorsal skin the next day after 5/6 nephrectomy. Blood pressure was measured by the indirect tail-cuff technique at 0 and 2 weeks after 5/6 nephrectomy. Mice were sacrificed at 2 weeks after 5/6 nephrectomy, and blood and tissue samples were collected.

Blood pressure monitoring. Blood pressure was measured in restrained, conscious mice by the indirect tail-cuff technique under unstressed conditions (BP-98A; Softron) at 0, 2, 4, 6, and 8 weeks after 5/6 nephrectomy (J. Lab. Clin. Med. 78:957-962). Before measurement, the mice were warmed with a heating pad for 5 minutes. A total of 7-10 readings were taken for each mouse, at 1- to 2-minute intervals.

Sample collection. Mice were sacrificed at 2, 4, and 8 weeks after 5/6 nephrectomy. The remnant kidney, lung, brain, heart, and liver were removed rapidly, snap-frozen in liquid nitrogen, and stored at −80° C. until examinations were performed. Each tissue was processed for histology, protein extraction, and RNA extraction. Blood samples were collected into chilled tubes for measurement of renal function parameters and into chilled tubes containing EDTA for Ang II measurements. Serum and plasma were separated by centrifugation (500 g for 10 minutes) and stored at −80° C. until measurement were performed. Serum creatinine, blood urea nitrogen (Mdk^{+/+}: sham, n=6; 2 weeks, n=10; 4 weeks, n=9; 8 weeks, n=8; Mdk^{-/-}: sham, n=7; 2 weeks, n=7; 4 weeks, n=6; 8 weeks, n=4), and Ang II concentrations (Mdk^{+/+}: sham, n=6; 2 weeks, n=6; 4 weeks, n=8; Mdk^{-/-}: sham, n=6; 2 weeks, n=7; 4 weeks, n=5) were measured by Mitsubishi BCL.

ACE activity assay. Lung ACE activity was measured in a fluorescence assay using a commercial ACE activity assay kit (Life Laboratory Company, Yamagata, Japan). Isolated lung tissue (Mdk^{+/+}: sham, n=5; 2 weeks, n=5; 4 weeks, n=5; Mdk^{-/-}: sham, n=5; 2 weeks, n=5; 4 weeks, n=4) was homogenized in an assay buffer and then clarified by centrifugation at 10,000 g for 15 minutes at 4.0° C. ACE activity against a synthetic substrate (benzyloxycarbonyl-phenyl alanyl-leucine) was determined using a colorimetric method. The product was measured fluorometrically at 355-nm excitation and 460-nm emission with a fluoro-colorimeter, as follows. For the assay, tissue samples were standardized to 1 μg protein/ml. Results were calculated as mU/mg protein. All data are reported as mean±SD. The measurements were performed in duplicate.

Histology and immunohistochemistry. The removed kidneys and lungs were fixed in 10% buffered formalin, embedded in paraffin, and then cut into 4-10.1 μm sections. The sections were stained with H&E, PAS, and Masson's trichrome. Another tissue sample was embedded in OCT compound (Sakura Finetek) and frozen in liquid nitrogen for immunostaining. Sections were cut to a thickness of 3 μm with a cryostat and fixed in acetone. High-power fields were used to examine the sections for evidence of focal sclerosis (J. Hypertens. 13:1785-1790, J. Lab. Clin. Med. 115:165-173). Glomerular sclerosis was assessed by semiquantitative score (grades 0 to +4) using the method of Raij et al. (Kidney Int. 26:137-143): grade 0, no sclerosis of glomeruli; grade 1, sclerosis of up to 25% of glomerulus; grade 2, sclerosis of 25%-50% of glomerulus; grade 3, sclerosis of 50%-75% of glomerulus; grade 4, sclerosis of 75%-100% of glomerulus. At least 50 glomeruli were evaluated under ×400 magnification and results averaged for each kidney (Mdk^{+/+}: 2 weeks, n=5; 4 weeks, n=4; 8 weeks, n=4; Mdk^{-/-}: 2 weeks, n=4; 4 weeks, n=3; 8 weeks, n=3). Immunostaining for ACE and MK was performed on buffered formalin-fixed tissues. Sections were deparaffinized, rehydrated, incubated in 3% hydrogen peroxide in methanol to block endogenous peroxidase, and washed in 10% normal goat serum (Dako) in PBS to block nonspecific binding. Subsequently, sections were incubated with mouse anti-ACE monoclonal antibody (dilution, 1:400; Chemicon International, Millipore) or anti-MK monoclonal antibody (dilution, 1:100) overnight at 4° C. as described previously (22), followed by a conjugate of polyclonal goat anti-mouse IgG antibody and HRP-labeled polymer (Histofine Simple Stain; Nichirei) for 1 hour at room temperature as a secondary antibody. The staining was visualized with 3-3′ diaminobenzidine (Nichirei) to produce a brown color. The sections were covered with 90% glycerol containing p-phenylenediamine and were examined by electron microscopy (H-7100; Hitachi). For double immunofluorescence staining of MK and thrombomodulin as a marker of the vascular endothelium, the cryosections of lungs were first incubated with chicken anti-human MK (dilution, 1:200) and then with rabbit anti-rat thrombomodulin (dilution, 1:1,000) (J. Immunol. 150:5633-5646), followed by incubation with FITC-labeled rabbit anti-chicken IgG (dilution, 1:160) and rhodamine-labeled goat anti-rabbit IgG (dilution, 1:320) as secondary antibodies. For immunofluorescence staining of macrophages and neutrophils, the cryosections of lungs or kidneys were first incubated with rat anti-mouse F4/80 antibodies (MCA497F; dilution, 1:50; AbD Serotec) or rat anti-mouse neutrophils (MCA771G, dilution, 1:200; AbD Serotec), followed by incubation with FITC-labeled goat anti-rat IgG F(ab)′ 2 (dilution, 1:160) or FITC-labeled rabbit anti-rat IgG (dilution, 1:160) as secondary antibodies. The lung and kidney section from each mouse was viewed under ×400 magnification, and then macrophages or neutrophils were counted from 10 fields and averaged. Values are mean±SD. HMVEC-L culture and treatments. HMVEC-L were used for the in vitro assay because they have previously been shown to express ACE (Microvasc. Res. 50:360-372). HMVEC-L (Takara Bio Inc.) were cultivated in EGM-2MV BulletKit medium (Takara Bio Inc.) at 37° C. in 5% CO2. HMVEC-L were grown as a monolayer in tissue culture plates coated with type I collagen. When the cells reached 70%-80% confluence, they were passaged with trypsin (0.025%)/EDTA (0.01%) and used within 4 passages, as recommended by the supplier. After reaching confluence, HMVEC-L were washed with PBS and cultured for 24 hours in essential basal medium containing 0.5% FBS. These cells were exposed to 100 ng/ml recombinant human MK (rh-MK) plus 20 μg/ml heparin or 50 ng/ml PMA (Sigma-Aldrich) in essential basal medium containing 0.5% FBS. PMA and the PKC inhibitor stock solutions were made in dimethyl sulfoxide. For PKC inhibition, BIS (Calbiochem) was added 1 hour before treatment with rh-MK plus heparin or PMA. Protein was extracted from HMVEC-L at the indicated time points after treatment. Cells were then lysed in RIPA buffer (50 mmol/l Tris-HCl, 150 mmol/l NaCl, 1% Nonidet P-40, 1% deoxycholic acid, and 0.05% sodium dodecyl sulfate) containing 0.25 mmol/l phenylmethylsulfonyl fluoride, kept on ice for 40 minutes, and then centrifuged at 15,000 g for 10 minutes at 4° C. The supernatants were then subjected to SDS-PAGE and Western blotting.

Ang II concentration in HMVEC-L. After reaching confluence, HMVEC-L were washed with PBS and cultured for 24 hours in essential basal medium containing 0.5% FBS. These cells were exposed to 100 ng/ml rh-MK plus 20 μg/ml heparin or 20 μg/ml heparin alone in essential basal medium containing 0.5% FBS for 36 hours. After stimulation, HMVEC-L were washed with PBS and exposed to 500 pM Ang I (Sigma-Aldrich) for 10 and 25 minutes. Supernatants were collected into chilled tubes, and then the Ang II concentration was measured as previously described (Hypertension. 39:129-134).

Western blot analysis. Mouse kidney, lung, brain, heart, and liver tissues were snap-frozen in liquid nitrogen for protein isolation. Western blot analysis was performed as described previously (Br. J. Cancer. 75:354-359). The blots were subsequently incubated with goat anti-human MK antibody (dilution, 1:1,000), monoclonal anti-β-actin antibody (dilution, 1:1,000; Sigma-Aldrich), mouse anti-ACE monoclonal antibody (dilution, 1:1,000; Chemicon International, Millipore), or rabbit anti-phospho-PKC antibody (dilution, 1:1,000; Cell Signaling Technology), followed by incubation with peroxidase-conjugated goat IgG, mouse IgG, or rabbit IgG (dilution, 1:5,000; Jackson Immunoresearch Laboratories Inc.). Western blot analysis of liver ANG was performed using the ANG-specific polyclonal antibody as described previously (J. Am. Soc. Nephrol. 12:431-439). Proteins were visualized with an enhanced chemiluminescence detection system (Amersham Pharmacia, GE Healthcare). The density of each band was measured using the public domain NIH Image program (http://rsb.info.nih.gov/nih-image/).

RNA preparation from mouse kidney and lung. Mouse kidney and lung tissues (15 mg) were immersed in RNAlater (Ambion, Applied Biosystems) for 1 day. The mixture was ground for 2 minutes with 5-mm tungsten carbide beads at a frequency of 20-25 Hz using a mixer-mill grinder according to the manufacturer's instructions (Tissuclyser; QIAGEN). The ground solution was then centrifuged for 3 minutes at 10,000 g to compact the debris, and the supernatant was treated according to the manufacturer's instructions. Total RNA was extracted using an RNeasy Mini Kit (QIAGEN). RNA concentrations were estimated using a spectrophotometer (Ultrospec 3300 pro; Amersham Biosciences, GE Healthcare).

Real-time PCR. First-strand cDNA was synthesized using the Quanti-Tect Reverse Transcription Kit (QIAGEN) according to the manufacturer's instructions, One microgram of total RNA was then reverse transcribed. To validate changes in gene expression, we performed real-time PCR analysis with an Applied Biosystems Prism 7500HT Sequence Detection System using TaqMan Gene Expression Assays according to the manufacturer's specifications (Applied Biosystems). Two microliters of cDNA samples was used for the PCR reaction. The TaqMan probes and primers were as follows. For mouse MK: forward,

5′-CAAGGGACCCTGAAGAAGGC-3′, and reverse,
5′-CTTTGGTCTTTGACTTGCTCTTGG-3; for ANG: forward,
5′-CTCGAACTCAAAGCAGGAGAGG-3′, and reverse,
5′-CGTAGATGGCGAACAGGAAGG-3′; for renin:
5′-TTGTTGCTCTGGAGTCCTTGC-3′, and reverse,
5′-CAGGATTTCCCGGACAGAAGG-3; for ACE: forward,
5′-ACCCAACCTCGATGTCACCA-3′, and reverse,
5′-GCGAGGTGAAGAATTCCTCTGA-3′; for Nox1: forward,
5′-TTGGCACAGTCAGTGAGGATG-3′, and reverse,
5′-AGATTTCAAGATGGAAGCAAAGGG-3′; for Nox2: forward,
5′-ACTTTCCATAAGATGGTAGCTTGG-3′, and reverse,
5′-GCATTCACACACCACTCAACG-3; and for Nox4: forward,
5′-ACCAGAATGAGGATCCCAGAAAG-3′, and reverse,
5′-GTAGAAGCTGTAACCATGAGGAAC-3′. 18S ribosomal RNA (assay identification number 4326317E), which was used as an endogenous control, was as assay-on-demand gene expression product (Applied Biosystems). The thermal cycler conditions were as follows: hold for 10 minutes at 95° C., followed by 2-step PCR consisting of 40 cycles at 95° C. for 15 seconds and 60° C. for 1 minute. The relative quantification of all targets was carried out using the comparative cycle threshold method (Methods. 25:402-408). The levels of gene expression were standardized with those of the 18S ribosomal RNA.

Statistics. Results are expressed as mean±SD. Statistical difference was assessed by a single-factor variance (ANOVA) followed by a 2-tailed unpaired t test, as appropriate. P values less than 0.05 were considered significant.

It will be obvious to those having skill in the art that many changes may be made in the above-described details of the preferred embodiments of the present invention.

The scope of the present invention, therefore, should be determined by the following claims.

What is claimed is:

1. A therapeutic method for treating mammalian kidney disorders, primary kidney disorders, hypertension that follows secondary kidney disorders such as diabetic nephropathy, and hypertension secondary to chronic kidney disease, said method comprising:
the step of suppressing midkine expression or activity.

2. The therapeutic method of claim 1, wherein said step of suppressing midkine expression or activity comprises
the step of preparing a midkine-based nucleic acid drug or a midkine neutralizing antibody; and
the step of administering said nucleic acid drug or neutralizing antibody.

3. The therapeutic method of claim 2, wherein the step of administering said nucleic acid drug or neutralizing antibody is a step of locally administering said nucleic acid drug or neutralizing antibody in the lung or the kidney.

4. The therapeutic method of claim 2, wherein the step of suppressing midkine expression or activity comprises
the step of preparing an angiotensin converting enzyme inhibitor or an AT1 receptor blocker; and
the step of administering said angiotensin converting enzyme inhibitor or AT1 receptor blocker.

5. The therapeutic method of claim 1, wherein the kidney disorders are any one of acute kidney failure, chronic kidney failure, end-stage kidney failure, or nephrosclerosis.

6. The therapeutic method of claim 1, wherein the mammalian is human.

7. A pharmacological composition comprising, as active ingredients, a therapeutically effective amount of a midkine-based nucleic acid drug or a midkine neutralizing antibody, in combination with one or more pharmacologically acceptable diluents, excipients and/or inert carriers.

8. The pharmacological composition of claim 7, wherein an angiotensin converting enzyme inhibitor or an AT1 receptor blocker is used in combination therewith.
